(19) United States
(12) Patent Application Publication
(10) Pub. No.: US 2007/0072911 A1
(43) Pub. Date: Mar. 29, 2007

(54) Indoles and azaindoles as antiviral agents

(76) Inventors: Salvatore Avolio, Casoria(Naples) (IT); Steven Harper, Albano Laziale (IT); Frank Narjes, Ariccia (IT); Barbara Pacini, Albano Laziale (IT); Michael Rowley, Axa (IT)

Correspondence Address: MERCK AND CO., INC, P O BOX 2000, RAHWAY, NJ, 07065-0907, US

(21) Appl. No.: 10/575,633

(22) PCT Filed: Oct. 8, 2004
(86) PCT No.: PCT/GB04/04306

§ 371 Date: Nov. 20, 2006

(30) Foreign Application Priority Data
Oct. 10, 2003 (GB) 0323845.8

(51) Int. Cl. A61K 31/4439; A61K 31/4245; A61K 31/4178; A61K 31/404; C07D 403/02; C07D 413/02

(52) U.S. Cl. 514/337; 514/415; 514/397; 546/277.4; 548/131; 548/312.1; 548/500; 514/364

(57) ABSTRACT

The present invention relates to indoles and azaindoles of formula (1): wherein X¹, X², X³, X⁴, A¹, Ar¹, Ar, n, p and q are as defined herein and pharmaceutically acceptable salts thereof, useful in the prevention and treatment of hepatitis C virus infections.

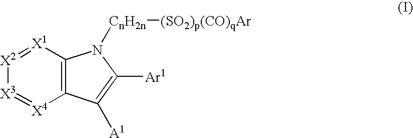

The present invention relates to indole and azaindole compounds, to pharmaceutical compositions containing them, to their use in the prevention and treatment of hepatitis C infections and to methods of preparation of such compounds and compositions.

Hepatitis C (HCV) is a cause of viral infections. There is as yet no adequate treatment for HCV infection but it is believed that inhibition of its RNA polymerase in mammals, particularly humans, would be of benefit. International patent applications WO 01/47883, WO 02/04425 and WO 03/000254 suggest fused ring compounds as possible inhibitors of HCV polymerase and illustrate thousands of possible benzimidazole derivatives that possess HCV polymerase inhibitory properties. However, these patent applications do not describe or reasonably suggest the preparation of any benzimidazole or azabenzimidazole substituted on all three available sites on the fused imidazole ring. WO 03/010140 and WO 03/010141 suggest further fused ring compounds as possible inhibitors of HCV polymerase and illustrate thousands of possible compounds all of which possess complex esterified side chains. None of these patent applications describe an indole or azaindole in which the indole nitrogen is substituted by an aromatic residue as described in the present application.

The present invention provides compounds of the formula (I): 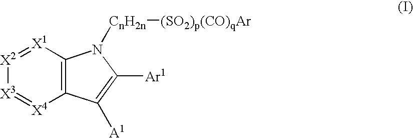
wherein:
Ar is a moiety containing at least one aromatic ring and possesses 5-, 6-, 9- or 10-ring atoms 0 to 3 of which may be N, O or S heteroatoms of which at most 1 will be O or S; which moiety may be optionally substituted by groups Q¹, Q² or Q³ wherein Q¹ is a hydroxy group, fluorine, chlorine, bromine or iodine atom or a C₁₋₆alkyl, C₁₋₆-alkyl substituted by not more than 5 fluorine atoms, C₁₋₆alkoxyl, C₁₋₆alkoxyl substituted by not more than 5 fluorine atoms, C₂₋₆alkenyl, C₂₋₆alkynyl, (CH₂)₀₋₃N(C₁₋₄alkyl)₂, nitro, cyano, nitrile, carboxyl, esterified carboxy wherein the esterifying moiety has up to 4 carbon atoms optionally substituted by not more than 5 fluorine atoms; or —SO₂(C₁₋₆alkyl), Q₂ is a fluorine, chlorine, bromine or iodine atom or a methyl, trifluoromethyl, methoxy, trifluoromethoxy or difluoromethoxy group, Q₃ is a fluorine, chlorine, bromine or iodine atom or a methyl, methoxy, trifluoromethoxy or difluoromethoxy group;
Ar¹ is a moiety containing at least one aromatic ring and possesses 5-, 6-, 9- or 10-ring atoms 0 to 3 of which may be N, O or S heteroatoms of which at most 1 will be O or S; which moiety may be optionally substituted by groups Q⁴, Q⁵ or Q⁶ wherein Q⁴ is a hydroxy group, fluorine, chlorine, bromine or iodine atom or a C₁₋₆alkyl, C₁₋₆alkyl substituted by not more than 5 fluorine atoms, C₁₋₆alkoxyl, C₁₋₆alkoxyl substituted by not more than 5 fluorine atoms, C₂₋₆alkenyl, C₂₋₆alkynyl, (CH₂)₀₋₃N(C₁₋₄alkyl)₂, nitro, cyano, nitrile, carboxyl, esterified carboxy wherein the esterifying moiety has up to 4 carbon atoms optionally substituted by not more than 5 fluorine atoms, Q⁵ is a fluorine, chlorine, bromine or iodine atom or a methyl, trifluoromethyl, methoxy, trifluoromethoxy or difluoromethoxy group, Q⁶ is a fluorine, chlorine, bromine or iodine atom or a methyl, methoxy, trifluoromethoxy or difluoromethoxy group;
X¹ is N or CR^{a}; X² is N or CR¹; X³ is N or CR²; X⁴ is N or CR^{b}; with the proviso that at least one of X² and X³ is not N; wherein R^{a} and R^{b} are independently selected from hydrogen, fluorine or chlorine or C₁₋₄alkyl, C₂₋₄alkenyl, C₁₋₄alkoxy, C₁₋₄alkyl or alkoxy optionally substituted by up to 6 fluorine atoms and/or a hydroxyl group;
n is 0, 1, 2, 3, 4, 5 or 6;
p+q is 0 or 1;
A¹ is C₁₋₆alkyl, C₂₋₆alkenyl, or C₁₋₆alkyl or C₂₋₆alkenyl substituted by C₁₋₄alkoxy or up to 5 fluorine atoms or a non-aromatic ring of 3 to 8 ring atoms which may contain a double bond and which may contain a O, S, SO, SO₂ or NH moiety and which may be optionally substituted by one or two alkyl groups of up to 2 carbon atoms or by 1 to 8 fluorine atoms;
one of R¹ and R² is a Het or is hydrogen, fluorine, chlorine or bromine atom or a C₁₋₄alkyl, C₂₋₄alkenyl, C₁₋₄alkoxy, C₁₋₄alkyl or alkoxy substituted by up to 5 fluorine atoms, nitrile, carboxy, C₁₋₄alkoxycarbonyl, C₁₋₄alkyl or C₂₋₄alkenyl substituted by a carboxy or C₁₋₄alkoxycarbonyl group, or a N³R⁴, SO₂NR³R⁴ or CONR³R⁴ group where R³ is hydrogen, C₁₋₄alkyl, SO₂R⁵ or COR⁵ and R⁴ is hydrogen, hydroxyl or C₁₋₄alkyl or R³ and R⁴ are alkylene linked to form a 5- or 6-membered ring, and R⁵ is C₁₋₄alkyl optionally substituted by up to 5 fluorine atoms;
   Het is a 5 or 6-membered aromatic ring of which 1, 2, 3 or 4 ring atoms may be selected from N, O, S with at most 1 being O or S which ring may be substituted by 1 or 2 groups selected C₁₋₄alkyl or hydroxy or tautomers thereof, or is 2-hydroxy-cyclobutene-3,4-dione:
the other of R¹ and R² is a hydrogen, fluorine or chlorine atom or C₁₋₄alkyl, C₂₋₄alkenyl, C₁₋₄alkoxy, C₁₋₄alkyl or alkoxy substituted by up to 6 fluorine atoms and optionally a hydroxyl;
or a pharmaceutically acceptable salt thereof.

The group CₙH₂ₙ may be straight or branched such as a —CH₂—, —(CH₂)₂—, —(CH₂)₃—, —(CH₂)₄—, —CH(CH₃)—, —CH₂—CH(CH₃)—, —CH(CH₃)—CH₂— or the like straight or branched butyl, pentyl or hexyl group. Most suitably the CₙH₂ₙ group is a —CH₂— group.

When used herein C₁₋₆alkyl means methyl, ethyl, 1-propyl, 2-propyl or a straight or branched butyl, pentyl or hexyl group. Particularly apt C₁₋₆alkyl groups are methyl, ethyl, propyl and butyl groups. Favoured alkyl groups are ethyl and methyl groups. The methyl group is the preferred alkyl group.

Most suitably a C₁₋₆alkyl group substituted by up to 5 fluorine atoms will include a CF₃, CHF₂ and/or CF₂ moiety. Favoured fluoroalkyl groups are the CF₃, CH₂F and CF₂CF₃ groups. The CF₃ group is the preferred fluoroalkyl group.

When used herein C₂₋₆alkenyl means a —CH═CH₂, —C(CH₃)═CH₂, —CH═C(CH₃), —C(CH₃)═C(CH₃) or straight or branched pentylene or hexylene groups.

When used herein C₁₋₆alkoxy and fluorinated C₁₋₆alkoxy are analogous to the alkyl and fluoroalkyl groups described above so that, for example, preferred groups include OCH₃, OCF₃ and OCHF₂ groups.

Favoured values for R^{a} and R^{b} independently include hydrogen, fluorine, methyl, methoxy and trifluoromethyl. Particularly apt values for R^{a} and R^{b} include hydrogen or fluorine. A preferred value for R^{a} is hydrogen. A preferred value for R^{b} is hydrogen.

The Ar moiety may contain a single aromatic ring or one aromatic ring to which a further aromatic or non-aromatic ring is fused.

Favoured values for Ar include optionally substituted 6-membered heteroaromatic groups with 1, 2 or 3 nitrogen ring atoms; unsubstituted or substituted 5-membered heteroaromatic groups with 1, 2, 3 or 4 nitrogen ring atoms; unsubstituted or substituted 5-membered heteroaromatic groups with one nitrogen ring atom and one oxygen or sulfur ring atoms; unsubstituted or substituted 5-membered heteroaromatic groups with two nitrogen atoms and one oxygen or sulfur atom. The optional substituents on such rings include one or two fluorine, chlorine, bromine, C₁₋₄alkyl, hydroxyl, C₁₋₄alkoxy or CF₃ groups of which methyl and hydroxyl are preferred.

Particularly apt values for Ar also include phenyl and substituted phenyl or the formula C₆H₂Q¹Q²Q³ of which phenyl, fluorophenyl, difluorophenyl, chlorophenyl, bromophenyl, dibromophenyl, methylphenyl, methoxyphenyl, methylsulfonylphenyl, carboxyphenyl, cyanophenyl, trifluoromethylphenyl and the like are preferred.

Ar is aptly an optionally substituted phenyl, pyridyl, imidazolyl, thiazolyl or oxadiazolyl group. The optional substituents on such groups include one or two fluorine, chlorine, bromine, C₁₋₆alkyl, hydroxyl, C₁₋₆alkoxy, CF₃, cyano, carboxyl, methylsulfonyl or (CH₂)₀₋₃N(C₁₋₄alkyl)₂ groups, of which methyl, fluoro, chloro, bromo, cyano, carboxyl, methylsulfonyl and CH₂N(CH₃)₂ are preferred.

Preferably Ar is a group selected from phenyl, methylphenyl, mono- or difluorophenyl, mono- or dichlorophenyl, mono- or dibromophenyl, cyanophenyl, carboxyphenyl, methylsulfonylphenyl, pyridyl, imidazolyl or methylthiazolyl. More particularly Ar is phenyl, 2-fluorophenyl, 4-fluorophenyl, 4-chlorophenyl, 3-methylphenyl, 4-methylphenyl, 3,5-dibromophenyl, 4-methylsulfonylphenyl, 3-carboxyphenyl, pyrid-2-yl, pyrid-3-yl, 2-methyl-1,3-thiazol-4-yl, 1H-imidazol-4-yl or 5-[(dimethylamino)methyl]-1,2,4-oxadiazol-3-yl.

Favourably n is 0, 1 or 2.

In one embodiment p is 1 and q is 0. In another embodiment p is 0 and q is 1. Alternatively p and q are both 0.

The Ar¹ moiety may contain a single aromatic ring or one aromatic ring to which a further aromatic or non-aromatic ring is fused.

Ar¹ is aptly phenyl, naphthyl, indolyl, tetrahydronaphthyl, pyridyl, imidazolyl, furyl, thienyl, pyrolidyl, oxazolyl, thiazolyl, pyrazolyl, pyridazolyl, triazolyl, oxadiazolyl, thiodiazolyl or quinonyl, any of which may be optionally substituted by group Q⁴, Q⁵ or Q⁶ as hereinbefore defined.

Favourably, Ar¹ is a pyridyl, furyl or thienyl group or a group of the formula C₆H₂Q⁴Q⁵Q⁶. One particularly favoured group Ar¹ is the pyridyl group. Other particularly favoured Ar¹ groups are optionally substituted phenyl groups of the formula C₆H₃Q¹Q² of which phenyl, fluorophenyl, chlorophenyl, hydroxyphenyl, trifluoromethylphenyl, methoxyphenyl, difluorophenyl, dichlorophenyl, {[isopropyl(methyl)amino]-methyl}phenyl and the like are preferred.

Preferably Ar¹ is phenyl, methoxyphenyl, fluorophenyl, chlorophenyl, hydroxyphenyl, pyridyl or {[isopropyl(methyl)amino]-methyl}. More particularly Ar¹ is phenyl, 4-methoxyphenyl, 2-fluorophenyl, 4-hydroxyphenyl, pyrid-2-yl or 2-(3-{[isopropyl(methyl)amino]-methyl}phenyl).

Particularly suitable groups A¹ include those groups of the formula: 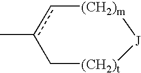
wherein m+t is 0, 1, 2, 3 or 4, preferably 1 or 2, the dotted line represents an optional bond and J is CH₂, O, S, SO, SO₂ or NH which group of the above formula may optionally be substituted by one or two methyl groups.

Favoured groups A¹ include cycloalkyl and cycloalkenyl groups of 5 or 6 ring members.

A preferred group A¹ is the cyclohexyl group.

Particularly apt compounds of this invention include those wherein one of R¹ and R² is a carboxy or —Y—CO₂H group wherein Y is CH₂, CH₂CH₂ or CH═CH group, or a pharmaceutically acceptable salt thereof.

A preferred group R¹ is the CO₂H group or a pharmaceutically acceptable salt thereof.

Favourably one of R¹ and R² is a hydrogen atom.

A favoured value for X₁ is CH.

A favoured value for X₄ is CH.

Favoured values for A¹ include non-aromatic rings. Such rings are aptly of 5 or 6 carbon atoms and which are saturated or monounsaturated. Preferred groups A¹ include cyclopentyl, cyclohexyl and cyclohexenyl groups.

Certain particularly suitable compounds of the invention are represented by the formula (II): 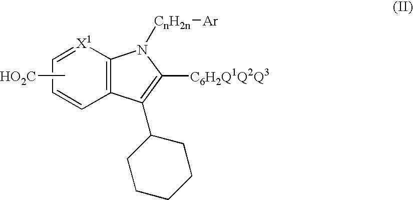
wherein n, X¹, Ar, Q¹, Q² and Q³ are as defined in relation to formula (I) or a pharmaceutically acceptable salt thereof.

In compounds of formulae (I) and (II) a favoured value for Q³ is H, a favoured value for n is 1 and a favoured value for X¹ is CH so that particularly apt compounds of the invention include those of formula (III): 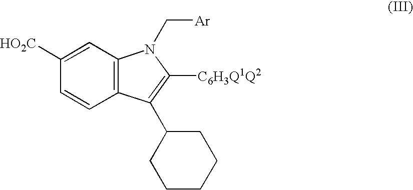
wherein Ar, Q¹ and Q² are defined in relation to formula (I), or a pharmaceutically acceptable salt thereof.

In certain apt compounds of formulae (II) and (III) Q² is hydrogen, fluorine chlorine, methyl, hydroxy, methoxy or trifluoromethyl. In certain apt compounds of formulae (II) and (III) Q¹ is hydrogen or fluorine. In certain preferred compounds of formulae (II) and (III) Q¹ is hydrogen and Q² is hydrogen, fluorine, methoxy or hydroxy.

The compounds of the formula (I) may be in the form of a pharmaceutically acceptable salt such as a sodium, potassium, calcium, magnesium or ammonium salt or a salt with a pharmaceutically acceptable organic base. If the compounds of the formula (I) also contain a group, the compound may be zwitterionic or in the form of a salt with a pharmaceutically acceptable acid such as hydrochloric, sulphuric, phosphoric, methane sulfonic and the like acid.

The present invention provides a process for the preparation of compounds of formula (I) and their salts which comprises the reaction of compounds of the formulae (IV) and (V): 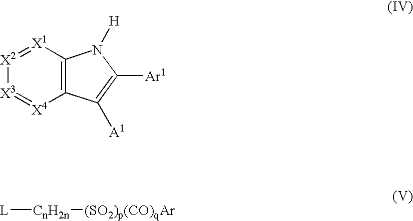
wherein X¹, X², X³, X⁴, A¹, Ar¹, Ar, n, p and q are as defined in relation to formula I and L is a good leaving group such as chlorine, bromine, iodine, methanesulfonate, tolyenesulfonate, triflate or the like.

In the compounds of formulae (IV) and (V) any reactions group that requires masking during the amidation reaction may be protected in conventional manner and the protecting group removed thereafter.

This principle of utilising protecting groups also applies to all other reactions described hereinafter. For example, if the desired compound of the formula I contains a CO₂H group, then the compound of the formula (IV) may contain a CO₂CH₃ group and the resulting compound of the formula (I) may be hydrolysed in conventional manner, for example with sodium hydroxide in aqueous methanol or BBr₃ in DCM to yield the compound containing the carboxylate or its sodium salt. Similarly the substituents on the core bicycle may be elaborated after the amidation reaction, for example if the desired compound of formula (I) contains a tetrazole group then the compound of formula (IV) may contain CN group and the resulting compound of formula (I) may be reacted with an azide.

In an alternative process the compounds of formula (I) may be prepared from the corresponding compound of the formula (VI): 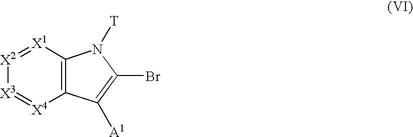
wherein X¹, X², X³, X⁴ and A¹ are as defined in relation to formula (I) and T is a CₙH₂ₙ(SO2)ₚ(CO)_{q}Ar group by reaction with Ar¹B(OH)₂ in the presence of a Pd[0] catalyst under conditions conventional for the Suzuki reaction, wherein n, p, q, Ar and Ar¹ are as defined in relation to formula (I).

The compound of formula (VI) wherein T is a CₙH₂ₙ(SO₂)ₚ(CO)_{q}Ar group can be prepared from the compound of formula (VI) wherein T is a hydrogen atom by reaction with a compound of formula (V).

Alternatively the compound of formula (VI) may be prepared by the reaction of NBS and the compound of the formula (VII): 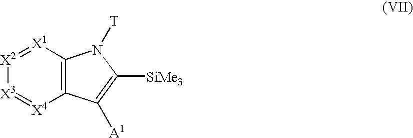
wherein T is CₙH₂ₙ(SO₂)ₚ(CO)_{q}Ar which may itself be prepared from the corresponding compound of formula (VII) wherein T is H by reaction with a compound of formula (V) under conventional alkylation conditions.

In an alternative synthesis the compounds of the formula (IV) may be prepared from the reaction of corresponding compounds of the formulae (VIII) and (IX):

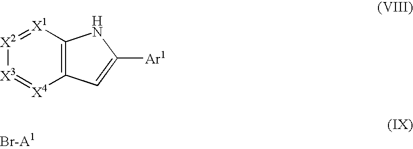

Similarly certain compounds of the formula (X) may be prepared by the reaction of a compound of the formula (VIII) with compounds of the formula (XI): 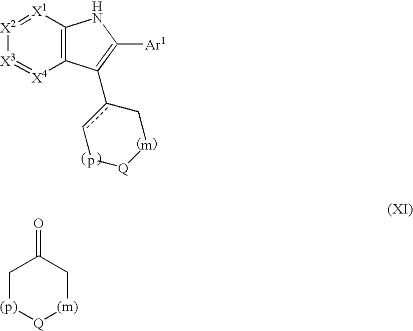
wherein Q is CH₂, NH, O, S, SO or SO₂ and m+p is 1 or 2 and where one or two optional substituents are selected from C₁₋₆ alkyl and hydroxyl and the dotted line is an optional bond; optionally followed by reduction of said optional bond.

The compounds of formula (X) may also be prepared by the reaction of the compounds of the formulae (XII) and (XIII): 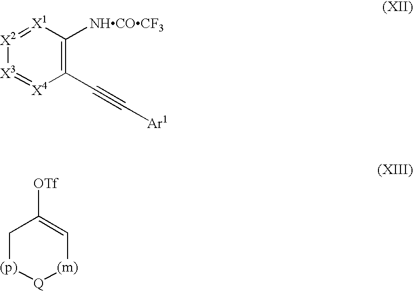
wherein Q, m and p are as defined in relation to formula (XI) in the presence of a Pd[0] catalyst optionally followed by reduction of the optional double bond.

The compound of the formula (XII) may be prepared from the compounds of the formulae (XIV) and (XV): 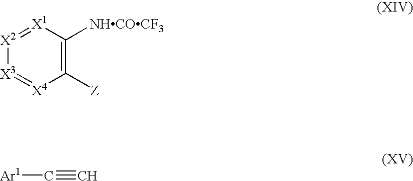
wherein Z is I, Br or OTf in the presence of a Pd[0] catalyst.

A further process for the preparation of the compounds of formula (VII) wherein T is hydrogen comprises the reaction of the compounds of the formulae: 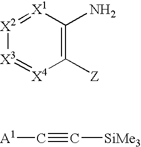
wherein Z is I, Br or OTf.

In addition, compounds of the formula (IV) may be prepared by the reaction of a hydrazine of the formula (XVIII): 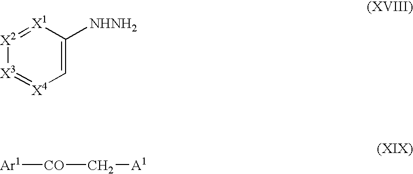
and a ketone of the formula (XIX).

This invention also provides compounds per se of formula (I) (II) or (III) except where Ar is phenyl and X² is an acidic function or salts and esters thereof.

The compounds of formulae (I)-(III) may be used for the inhibition of HCV polymerase and so may be used for the manufacture of medicaments which may be used to treat HCV infection.

Accordingly this invention provides a pharmaceutical composition comprising a compound of the formula (I) as hereinbefore described as a pharmaceutically acceptable salt thereof together with a pharmaceutically acceptable carrier.

The invention also provides pharmaceutical compositions comprising one or more compounds of this invention in association with a pharmaceutically acceptable carrier. Preferably these compositions are in unit dosage forms such as tablets, pills, capsules, powders, granules, sterile parenteral solutions or suspensions, metered aerosol or liquid sprays, drops, ampoules, auto-injector devices or suppositories; for oral, parenteral, intranasal, sublingual or rectal administration, or for administration by inhalation or insufflation. For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the present invention. Typical unit dosage forms contain from 1 to 100 mg, for example 1, 2, 5, 10, 25, 50 or 100 mg, of the active ingredient. The tablets or pills of the novel composition can be coated or otherwise compounded to provide a dosage form affording the advantage of prolonged action.

The liquid forms in which the novel compositions of the present invention may be incorporated for administration orally or by injection include aqueous solutions, suitably flavoured syrups, aqueous or oil suspensions, and flavoured emulsions with edible oils such as cottonseed oil, sesame oil, coconut oil or peanut oil, as well as elixirs and similar pharmaceutical vehicles. Suitable dispersing or suspending agents for aqueous suspensions include synthetic and natural gums such as tragacanth, acacia, alginate, dextran, sodium carboxymethylcellulose, methylcellulose, polyvinyl-pyrrolidone or gelatin.

In the treatment of infection due to hepatitis C, a suitable dosage level is about 0.01 to 250 mg/kg per day, preferably about 0.05 to 100 mg/kg per day, and especially about 0.05 to 5 mg/kg per day. The compounds may be administered on a regimen of 1 to 4 times per day. Most suitably the administration is orally using a unit done as previously indicated.

In a further aspect this invention provides the use of a compound of formula (I) or a pharmaceutically acceptable salt thereof in the manufacture of a medicament for the treatment of infection by hepatitis C virus. Most suitably the medicament is in unit dose form adapted for oral administration as indicated hereinbefore.

In another aspect this invention provides the use of a compound of formula (I) or a pharmaceutically acceptable salt thereof for the treatment of infection by hepatitis C virus in a mammal and preferably in a human. Most suitably the treatment is effected by oral administration of a unit dose form as indicated hereinbefore.

The following Examples are illustrative of this invention.

The compounds of the invention were tested for inhibitory activity against the HCV RNA dependent RNA polymerase (NS5B) in an enzyme inhibition assay (example i)) and an cell based sub-genomic replication assay (described in example ii)). The compounds generally have IC50's below 0.5 μM in the enzyme assay and EC50's typically below 20 μM in the cell based assay.

i) In-Vitro HCV NS5B Enzyme Inhibition Assay

WO 96/37619 describes the production of recombinant HCV RdRp from insect cells infected with recombinant baculovirus encoding the enzyme. The purified enzyme was shown to possess in vitro RNA polymerase activity using RNA as template. The reference describes a polymerisation assay using poly(A) and oligo(U) as a primer or an heteropolymeric template. Incorporation of tritiated UTP or NTPs is quantified by measuring acid-insoluble radioactivity. The present inventors have employed this assay to screen the various compounds described above as inhibitors of HCV RdRp.

Incorporation of radioactive UMP was measured as follows. The standard reaction (50 μl) was carried out in a buffer containing 20 mM tris/HCl pH 7.5, 5 mM MgCl₂, 1 mM DTT, 50 mM NaCl, 0.03% N-octylglucoside, 1 μCi [³H-UTP (40 Ci/mmol, NEN), 10 μM UTP and 10 μg/ml poly(A) or 5 μM NTPs and 5 μg/ml heteropolymeric template. Oligo(U)₁₂ (1 μg/ml, Genset) was added as a primer in the assay working on Poly(A) template. The final NS5B enzyme concentration was 5 nM. The order of assembly was: 1) compound, 2) enzyme, 3) template/primer, 4) NTP. After 1 h incubation at 22° C. the reaction was stopped by adding 50 μl of 20% TCA and applying samples to DE81 filters. The filters were washed thoroughly with 5% TCA containing 1M Na₂HPO₄/NaH₂PO₄, pH 7.0, rinsed with water and then ethanol, air dried, and the filter-bound radioactivity was measured in the scintillation counter. Carrying out this reaction in the presence of various concentrations of each compound set out above allowed determination of IC₅₀ values by utilising the formula:
% Residual activity=100/(1+[I]/IC₅₀)^{s}
where [I] is the inhibitor concentration and “s” is the slope of the inhibition curve.
ii) Cell Based HCV Replication Assay

Cell clones that stably maintain subgenomic HCV replicon were obtained by transfecting Huh-7 cells with an RNA replicon identical to I₃₇₇neo/NS3-3′/wt described by Lohmann et al. (1999) (EMBL-genbank No. AJ242652), followed by selection with neomycin sulfate (G418). Viral replication was monitored by measuring the expression of the NS3 protein by an ELISA assay performed directly on cells grown in 96 wells microtiter plates (Cell-ELISA) using the anti-NS3 monoclonal antibody 10E5/24 (we have first described the assay in our replicon patent WO 0259321 A2). Cells were seeded into 96 well plates at a density of 10⁴ cells per well in a final volume of 0.1 ml of DMEM/10% FCS. Two hours after plating, 50 μl of DMEM/10% FCS containing a 3× concentration of inhibitor were added, cells were incubated for 96 hours and then fixed for 10′ with ice-cold isopropanol. Each condition was tested in duplicate and average absorbance values were used for calculations. The cells were washed twice with PBS, blocked with 5% non-fat dry milk in PBS+0.1% Triton X100+0.02% SDS (PBSTS) and then incubated o/n at 4° C. with the 10E5/24 mab diluted in Milk/PBSTS. After washing 5 times with PBSTS, the cells were incubated for 3 hours at room temperature with Fc specific anti-mouse IgG conjugated to alkaline phosphatase (Sigma), diluted in Milk/PBSTS. After washing again as above, the reaction was developed with p-Nitrophenyl phosphate disodium substrate (Sigma) and the absorbance at 405/620 nm read at intervals. For calculations, we used data sets where samples incubated without inhibitors had absorbance values comprised between 1 and 1.5. The inhibitor concentration that reduced by 50% the expression of NS3 (IC₅₀) was calculated by fitting the data to the Hill equation,
Fraction inhibition=1-(Ai−b)/(A₀−b)=[I]ⁿ/([I]ⁿ+IC₅₀)
where:

Ai=absorbance value of HB110 cells supplemented with the indicated inhibitor concentration.

A₀=absorbance value of HB110 cells incubated without inhibitor.

b=absorbance value of Huh-7 cells plated at the same density in the same microtiter plates and incubated without inhibitor.

n=Hill coefficient.

General Synthetic Procedures

All solvents were obtained from commercial sources (Fluka, puriss.) and were used without further purification. With the exception of routine deprotection and coupling steps, reactions were carried out under an atmosphere of nitrogen in oven dried (110° C.) glassware. Organic extracts were dried over sodium sulfate, and were concentrated (after filtration of the drying agent) on rotary evaporatorators operating under reduced pressure. Flash chromatography was carried out on silica gel following published procedure (W. C. Still et al., J. Org. Chem. 1978, 43, 2923) or on commercial flash chromatography systems (Biotage corporation and Jones Flashmaster) utilising pre-packed columns.

Reagents were usually obtained directly from commercial suppliers (and used as supplied) but a limited number of compounds from in-house corporate collections were utilised. In the latter case the reagents are readily accessible using routine synthetic steps that are either reported in the scientific literature or are known to those skilled in the art. ¹H nmr spectra were recorded on Bruker A M series spectrometers operating at (reported) frequencies between 300 and 600 MHz and unless otherwise stated were recorded at 300K. Chemical shifts (δ) for signals corresponding to non-exchangeable protons (and exchangeable protons where visible) are recorded in parts per million (ppm) relative to tetramethylsilane and are measured using the residual solvent peak as reference. Signals are tabulated in the order: multiplicity (s, singlet; d, doublet; t, triplet; q, quartet; m, multiplet; br, broad, and combinations thereof); coupling constant(s) in hertz; number of protons. Mass spectral (MS) data were obtained on a Perkin Elmer API 100 operating in negative (ES⁻) or positive (ES⁺) ionization mode and results are reported as the ratio of mass over charge (m/z) for the parent ion only. Preparative scale HPLC separations were carried out on a Waters Delta Prep 4000 separation module, equipped with a Waters 486 absorption detector or on a Thermoquest P4000 equipped with a UV1000 absorption detector. In all cases compounds were eluted with linear gradients of water and acetonitrile both containing 0.1% TFA using flow rates between 15 and 25 mL/min.

The following abbreviations are used in the examples, the schemes and the tables:
DMF: dimethylformamide; DMSO: dimethylsulfoxide; eq.: equivalent(s); AcOEt: ethyl acetate; Eₜ₂O: diethyl ether; MeCN: acetonitrile; h: hour(s); Me: methyl; EtOH: ethanol; min: minutes; Ph: phenyl; HPLC: reversed phase high-pressure liquid chromatography; TFA: trifluoroacetic acid; THF: tetrahydrofuran; MeOH: methanol; TFAA: trifluoroacetic anhydride
   Representative Synthetic Procedures

Compounds from the invention were prepared by functionalisation of an N-unsubstituted 2-aryl-3-cycloalkyl indole carboxylic ester as outlined in scheme 1.

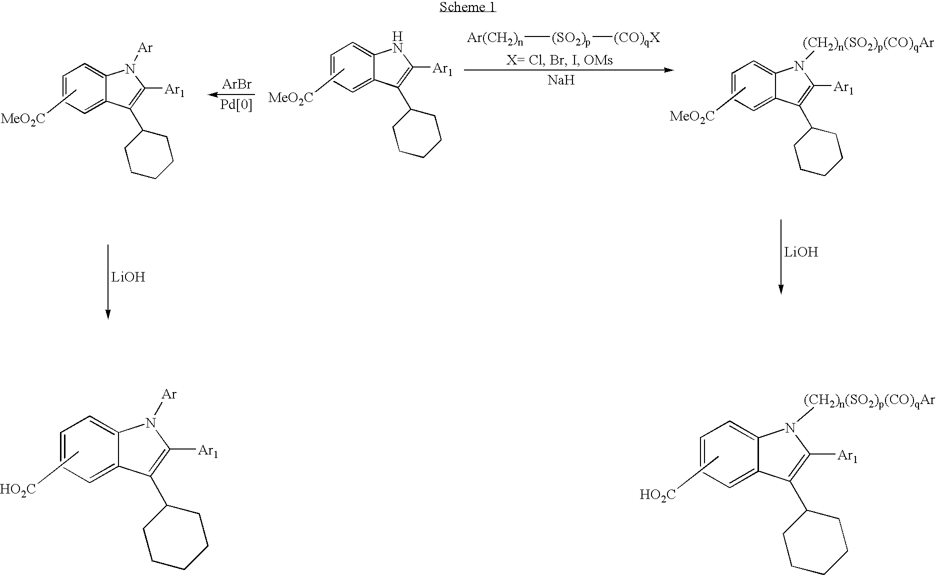

Several routes are reported in the literature that may be used to access 2-aryl-3-cycloalkyl indole carboxylic ester. Useful references include: Nanomoto et al, J. Chem. Soc. Perkin I, 1990, III; Freter, J. Org. Chem., 1975, 40, 2525; Cacchi et al, Eur. J. Org. Chem., 2002, 2671; Ujjainwalla, Tetrahedron Lett., 1998, 39, 5355; Wang et al, J. Org. Chem., 2000, 65, 1889; Larock, J. Org. Chem., 1998, 63, 7652; Kelly et al, J. Org. Chem., 1996, 61, 4623; and Cacchi, Tetrahedron Lett., 1992, 33, 3915. The synthetic route used in the current work is shown in scheme 2.

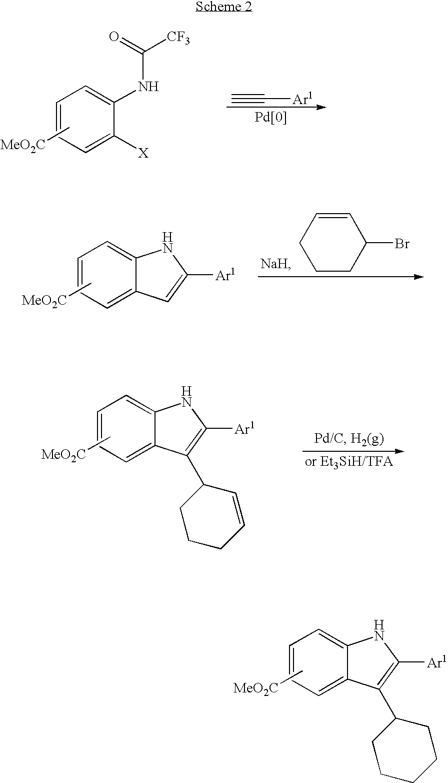

### EXAMPLE 1

### 1-benzyl-3-cyclohexyl-2-phenyl-1H-indole-5-carboxylic acid

### Step 1: methyl 3-iodo-4-[(trifluoroacetyl)amino]benzoate

A solution (0.26 M) of the substrate in dry THF was treated dropwise at 0° C. with TFAA (2 eq). The mixture was stirred for 10 min, then adjusted to pH 8 by addition of saturated aqueous NaHCO₃. The mixture was extracted with AcOEt and the organic phase was washed with brine then dried. Removal of the solvent gave the title compound (100%) as a solid.

¹H NMR (300 MHz, DMSO-d₆) δ 3.89 (s, 3H), 7.57 (d, J 8.2 Hz, 1H), 8.03 (dd, J 1.9, 8.2 Hz, 1H), 8.44 (d, J 1.9 Hz, 1H)

### Step 2: methyl 3-(phenylethynyl)-4-[(trifluoroacetyl)amino]benzoate

A solution (0.2 M) of methyl 3-iodo-4-[(trifluoroacetyl)amino]benzoate in a 4:1 mixture of diethylamine/DMF was treated with phenyl acetylene (1.2 eq) and Pd(PPh₃)₂Cl₂ (0.02 eq). The solution was stirred for 5 min. then treated with CuI (0.01 eq). After 12 h the reaction was diluted with Et₂O aqueous HCl (1 N) then the organic phase was separated and washed with brine and dried. Removal of the solvent gave a residue that was purified by flash chromatography on silica gel (9:1 AcOEt:petroleum ether) to afford the title compound (71%) as a solid.

¹H NMR (400 MHz, DMSO-d₆) δ 3.89 (s, 3H), 7.42-7.50 (m, 3H), 7.50-7.57 (m, 2H), 7.68 (d, J 8.4 Hz, 1H), 8.04 (dd, J 2.0, 8.4 Hz, 1H), 8.16 (d, J 2.0 Hz, 1H), 11.45 (s, 1H)

### Step 3: methyl 3-cyclohex-1-en-1-yl-2-phenyl-1H-indole-5-carboxylate

A solution (0.2 M) of methyl 3-(phenylethynyl)-4-[(trifluoroacetyl)amino]benzoate in MeCN was treated with cyclohex-1-en-1-yl trifluoromethanesulfonate (1.0 eq) and K₂CO₃ (5.0 eq). Pd(PPh₃)₄ (0.05 eq) was added and the mixture was stirred at room temperature for 2 h. After dilution with Et₂O and aqueous HCl (1 N) the organic layer was separated and washed with water and brine then dried. Removal of the solvent afforded the title compound (80%) as a solid.

¹H NMR (400 MHz, DMSO-d₆) δ 1.70 (s, 4H), 2.08 (s, 2H), 2.16 (s, 2H), 3.84 (s, 3H), 5.83 (s, 1H), 7.36 (t, J 7.4 Hz, 1H), 7.35 (d, J 8.4 Hz, 1H), 7.49 (t, J 7.4 Hz, 2H), 7.70 (d, J 7.4 Hz, 2H), 7.74 (dd, J 1.5, 8.4 Hz, 1H), 8.14 (s, 1H), 11.73 (s, 1H).

### Step 4: methyl 3-cyclohexyl-2-phenyl-1H-indole-5-carboxylate

A solution (0.05 M) of methyl 3-cyclohex-1-en-1-yl-2-phenyl-1H-indole-5-carboxylate in MeOH was treated with 50 wt % Pd/C (10% by weight) and ammonium formate (4.0 eq). The mixture was stirred under reflux for 5 h then cooled and filtered. The filtrate was treated with fresh catalyst and and ammonium formate as above and heated under reflux for 10 h. The cooled solution was filtered and concentrated to give the title compound as an oil.

¹H NMR (400 MHz, DMSO-d₆) δ 1.20-1.45 (m, 3H), 1.70-1.90 (m, 5H), 1.90-2.06 (m, 2H), 2.76-3.04 (m, 1H), 3.88 (s, 3H), 7.41 (d, J 8.4 Hz, 1H), 7.41-7.46 (m, 1H), 7.49-7.58 (m, 4H), 7.72 (d, J 8.4 Hz, 1H), 8.42 (s, 1H), 11.55 (s, 1H).

### Step 5: 1-benzyl-3-cyclohexyl-2-phenyl-1H-indole-5-carboxylic acid

A solution (0.06 M) of methyl 3-cyclohexyl-2-phenyl-1H-indole-5-carboxylate in dry THF was treated with NaH (1.4 eq) then stirred at room temperature for 0.5 h. Benzyl bromide (1.15 eq) was added and the mixture was stirred for 5 h. The solvent was removed, and the residue was diluted to 0.05 M with CH₂Cl₂. BBr₃ (3.0 eq) was added and the mixture was stirred for 0.5 h then concentrated in vacuo. The residue was treated with H₂0 then purified by HPLC (stationary phase: Waters Symmetry C₁₈ 19×100 mm; mobile phase: 50% to 100% MeCN in H₂O over 10 min) to give the title compound (16%) as a solid.

¹H NMR (300 MHz, DMSO-d₆) δ 1.09-1.39 (m, 3H), 1.60-1.99 (m, 7H), 2.55-2.72 (m, 1H), 5.25 (s, 2H), 6.81 (d, J 6.5 Hz, 2H), 7.12-7.29 (m, 3H), 7.31-7.39 (m, 2H), 7.42 (d, J 8.6 Hz, 1H), 7.46-7.56 (m, 3H), 7.72 (d, J 8.6 Hz, 1H), 8.44 (s, 1H), 12.51 (br s, 1H); MS (ES⁺) m/z 410 (M+H)⁺

### EXAMPLE 2

### 1-benzyl-3-cyclohexyl-2pyridin-2-yl-1H-indole-6-carboxylic acid

### Step 1: methyl 3-amino-4-hydroxybenzoate

A solution (0.2 M) of acetyl chloride (3.0 eq) in MeOH was prepared at 0° C. then allowed to warm to 20° C. 3-amino-4-hydroxybenzoic acid (1.0 eq) was added and the mixture was heated under reflux for 12 h then cooled and concentrated in vacuo. The residue was triturated with H₂O and dried to afford the title compound (99%) as a solid.

¹H NMR (300 MHz, DMSO-d₆) δ 3.83 (s, 3H), 7.15 (d, J 8.5 Hz, 1H), 7.79 (dd, J 2.1, J 8.5 Hz, 1H), 7.93 (d, J 2.1 Hz, 1H), 11.65 (br s, 1H)

### Step 2: methyl 4-hydroxy-3-[(trifluoroacetyl)amino]benzoate

A solution (0.2 M) of methyl 3-amino-4-hydroxybenzoate in THF was cooled to 0° C. and treated dropwise with trifluoroacetic anhydride (2.0 eq). The mixture was stirred at 0° C. for 2 h then at 20° C. for 1 h. The pH was adjusted to 7.5 by addition of saturated aqueous NaHCO₃ and the solution was extracted with AcOEt. The organic layer was washed with brine and dried, then concentrated to afford the title compound (87%) as a solid.

¹H NMR (400 MHz, DMSO-d₆) δ 3.82 (s, 3H), 7.02 (d, J 8.5 Hz, 1H), 7.77 (dd, J 2.1, J 8.5 Hz, 1H), 7.97 (d, J 2.1 Hz, 1H), 10.82 (br s, 1H)

### Step 3: methyl 3-[(trifluoroacetyl)amino]-4-{[(trifluoromethyl)sulfonyl]oxy}benzoate

A solution (0.8 M) of methyl 4-hydroxy-3-[(trifluoroacetyl)amino]benzoate in dry pyridine was cooled to 0° C. and treated dropwise with trifluoromethanesulfonyl anhydride (1.15 eq). The mixture stirred for 1 h at 20° C. then diluted with H₂O and AcOEt. The organic layer was separated and washed with aqueous HCl (1 N) and brine then dried. Removal of the solvent afforded a residue that was purified by flash chromatography (1:9 AcOEt:petroleum ether eluent) to afford the title compound (64%) as a solid.

¹H NMR (300 MHz, DMSO-d₆) δ 3.92 (s, 3H), 7.82 (d, J 8.7 Hz, 1H), 8.11 (dd, J 2.2, J 8.7 Hz, 1H), 8.17 (d, J 2.2 Hz, 1H), 11.81 (s, 1H)

### Step 4: methyl 2-pyridin-2-yl-1H-indole-6-carboxylate

A solution (0.2 M) of methyl 3-[(trifluoroacetyl)amino]-4-{[(trifluoromethyl)sulfonyl]oxy)benzoate in dry DMF was treated with 2-ethynylpyridine (2.0 eq) and tetramethylguanidine (10.0 eq). Pd(PPh₃₎₂Cl₂ (0.1 eq) and CuI (0.1 eq) were added and the mixture was stirred at room temperature for 0.5 h. The temperature was increased to 100° C. for 8 h then the mixture was cooled and filtered through celite with Et₂O. The filtrate was washed with aqueous HCl (1 N) and brine then dried. Removal of the solvent gave a residue that was purified by flash chromatography on silica gel (15:85 AcOEt:petroleum ether) to afford the title compound as a solid.

¹H NMR (400 MHz, DMSO-d₆) δ 3.88 (s, 3H), 7.27 (s, 1H), 7.43-7.52 (m, 1H), 7.64 (d, J 8.4 Hz, 1H), 7.69 (d, J 8.4 Hz, 1H), 7.93 (dt, J 1.6, 7.7 Hz, 1H), 8.08 (d, J 7.7 Hz, 1H), 8.16 (s, 1H), 8.70 (d, J 4.9 Hz, 1H), 12.12 (s, 1H)

### Step 5: methyl 3-cyclohex-2-en-1-yl-2-pyridin-2-yl-1H-indole-6-carboxylate

A solution (0.06 M) of methyl 2-pyridin-2-yl-1H-indole-6-carboxylate in DMF was cooled to 0° C. and treated with NaH (1.2 eq). The mixture was stirred at room temperature for 1 h then treated dropwise at 0° C. with a solution (0.5 M) of 3-bromocyclohexene in DMF. After stirring for 1 h in DMF the mixture was diluted with AcOEt and aqueous HCl (1 N). The organic phase was separated and washed with brine then dried. Removal of the solvent gave a residue that was purified by flash chromatography on silica gel (1:9 AcOEt:petroleum ether) to afford the title compound (18%) as a solid.

¹H NMR (400 MHz, DMSO-d₆) δ 1.62-1.81 (m, 1H), 1.83-1.98 (m, 2H), 2.00-2.33 (m, 3H), 3.88 (s, 3H), 4.28-4.48 (m, 1H), 5.75 (d, J 9.8 Hz, 1H), 5.86-5.96 (m, 1H), 7.42 (dd, J 4.8, 7.5 Hz, 1H), 7.58 (dd, J 1.4, 8.4 Hz, 1H), 7.76 (d, J 8.4 Hz, 1H), 7.80 (d, J 7.5 Hz, 1H), 7.99 (dt, J 1.7, 7.5 Hz, 1H), 8.10 (d, J 1.4 Hz, 1H), 8.76 (d, J 4.8 Hz, 1H), 11.78 (s, 1H)

### Step 6: methyl 3-cyclohexyl-2-pyridin-2-yl-1H-indole-6-carboxylate

A solution (0.015 M) of methyl 3-cyclohex-2-en-1-yl-2-pyridin-2-yl-1H-indole-6-carboxylate in MeOH was treated with 20% by weight of Pd/C (10 wt %) and stirred for 12 h under an atmosphere of hydrogen gas. The solution was purged with nitrogen then filtered. The filtrate was concentrated to afford the title compound (94%) as a solid.

¹H NMR (400 MHz, DMSO-d₆) δ 1.33-1.49 (m, 3H), 1.70-1.93 (m, 5H), 1.97-2.13 (m, 2H), 3.30-3.46 (m, 1H), 3.88 (s, 3H), 7.43 (dd, J 4.7, 7.5 Hz, 1H), 7.60 (dd, J 1.5 8.4 Hz, 1H), 7.72 (d, J 7.5 Hz, 1H), 7.92 (d, J 8.4 Hz, 1H), 7.99 (dt, J 1.6, 7.5 Hz, 1H), 8.10 (d, J 1.5 Hz, 1H), 8.77 (d, J 4.7 Hz, 1H), 11.69 (s, 1H)

### Step 7: 1-benzyl-3-cyclohexyl-2-pyridin-2-yl-1H-indole-6-carboxylic acid

A solution (0.06 M) of methyl 3-cyclohexyl-2-pyridin-2-yl-1H-indole-6-carboxylate in dry THF was treated with NaH (1.4 eq). The mixture was stirred at room temperature for 0.5 h. Benzyl bromide (1.15 eq) was added and the mixture was stirred for 5 h. The mixture was diluted with NH₄Cl and extracted with AcOEt. The organic layer was washed with brine and dried then concentrated to give a residue that was dissolved in a 4:1 mixture of THF:H₂O (0.07 M) and treated with LiOH.H₂O (4 eq). The mixture was stirred at 50° C. for 6 h then the solvent was removed. The residue was acidified with aqueous HCl (1 N) and AcOEt, and the organic layer was washed with brine and dried. The residue obtained after removal of the solvent was purified by HPLC (stationary phase: Waters Symmetry C₁₈ 19×100 mm; mobile phase: 10% MeCN to 100% MeCN in H₂O over 12 min) to give the title compound (60%) as a solid. ¹H NMR (400 MHz, DMSO-d₆) δ 1.14-1.46 (m, 3H), 1.62-1.86 (m, 5H), 1.86-2.04 (m, 2H), 2.64-2.80 (m, 1H), 5.49 (s, 2H), 6.84 (d, J 6.9 Hz, 2H), 7.08-7.22 (m, 3H), 7.41-7.51 (m, 2H), 7.65 (d, J 8.4 Hz, 1H), 7.86-7.97 (m, 2H), 8.01 (s, 1H), 8.76 (d, J 4.3 Hz, 1H); MS (ES⁺) m/z 411 (M+H)⁺

### EXAMPLE 3

### 1-benzyl-3-cyclohexyl-2-(4-methoxyphenyl)-1H-indole-6-carboxylic acid

### Step 1: methyl 2-(4-methoxyphenyl)-1H-indole-6-carboxylate

Using the procedure described in example 2 step 4, treatment of 3-[(trifluoroacetyl)amino]-4-{[(trifluoromethyl)sulfonyl]oxy}benzoate with 4-ethynylanisole (2.0 eq) gave a residue that was purified by flash chromatography on silica gel (3:7 AcOEt:petroleum ether) to afford the title compound (51%) as a solid.

¹H NMR (400 MHz, DMSO-d₆) δ 3.82 (s, 3H), 3.85 (s, 3H), 6.88 (d, J 1.1 Hz, 1H), 7.07 (d, J 8.7 Hz, 2H), 7.57 (d, J 8.4 Hz, 1H), 7.61 (dd, J 1.1, J 8.4 Hz, 1H), 7.83 (d, J 8.7 Hz, 2H), 8.02 (br s, 1H), 11.79 (s, 1H)

### Step 2: methyl 3-cyclohex-2-en-1-yl-2-(4-methoxyphenyl)-1H-indole-6-carboxylate

Using the procedure described in example 2 step 5, treatment of 2-(4-methoxyphenyl)-1H-indole-6-carboxylic acid with NaH (1.1 eq) and 3-bromocyclohexene (1.3 eq) gave a residue that was purified by flash chromatography on silica gel (1:9 AcOEt:petroleum ether) to afford the title compound (25%) as a solid.

¹H NMR (300 MHz, DMSO-d₆) δ 1.55-1.75 (m, 1H), 1.85-2.02 (m, 3H), 2.05-2.30 (m, 2H), 3.65-3.77 (m, 1H), 3.84 (s, 3H), 3.86 (s, 3H), 5.62-5.72 (m, 1H), 5.80-5.91 (m, 1H), 7.13 (d, J 8.8 Hz, 2H), 7.51 (d, J 8.8 Hz, 2H), 7.57 (dd, J 8.5, 1.4 Hz, 1H), 7.64 (d, J 8.5 Hz, 1H), 8.00 (d, J 1.4 Hz, 1H), 11.49 (s, 1H)

### Step 3: methyl 3-cyclohexyl-2-(4-methoxyphenyl)-1H-indole-6-carboxylate

Following the procedure described in example 2 step 6, treatment of 3-cyclohex-2-en-1-yl-2-(4-methoxyphenyl)-1H-indole-6-carboxylic acid with Pd/C gave the title compound (91%) as a solid.

¹H NMR (400 MHz, DMSO-d₆) δ 1.15-1.45 (m, 3H), 1.65-1.85 (m, 5H), 1.85-2.10 (m, 2H), 2.75-2.95 (m, 1H), 3.83 (s, 3H), 3.85 (s, 3H), 7.12 (d, J 8.7 Hz, 2H), 7.56 (d, J 8.7 Hz, 2H), 7.58 (dd, J 1.7, 8.4 Hz, 1H), 7.81 (d, J 8.4 Hz, 1H), 7.97 (d, J 1.7 Hz, 1H), 11.39 (s, 1H)

### Step 4: 1-benzyl-3-cyclohexyl-2-(4-methoxyphenyl)-1H-indole-6-carboxylic acid

A solution (0.04 M) of 3-cyclohexyl-2-(4-methoxyphenyl)-1H-indole-6-carboxylic acid in DMF was treated with NaH (1.5 eq) and the mixture was stirred for 1 h at room temperature. Benzylbromide (1.8 eq) was added and the mixture was stirred at room temperature for 1 h. After dilution with AcOEt the organic layer was washed with HCl (1 N) and brine then dried. Removal of the solvent gave a residue that was purified by flash chromatography on silica gel (10:90 AcOEt:PE) then diluted to 0.03 M with 1:1 THF:H₂O. LiOH.H₂O (10 eq) were added and the mixture was stirred at 40° C. for 3 days. After removal of the solvent, the residue was treated with aqueous HCl (1 N) then filtered and purified by HPLC (stationary phase: Waters Symmetry C₁₈ 19×100 mm; mobile phase: 50% to 100% MeCN in H₂O over 10 min; retention time: 8.0 min) to give the title compound (52%) as a solid.

¹H NMR (400 MHz, DMSO-d₆) δ 1.13-1.33 (m, 3H), 1.63-1.70 (m, 1H), 1.70-1.80 (m, 4H), 1.83-1.90 (m, 2H), 2.56-2.62 (m, 1H), 3.80 (s, 3H), 5.26 (s, 2H), 6.83 (d, J 6.8 Hz, 2H), 7.04 (d, J 8.4 Hz, 2H), 7.17 (t, J 6.8 Hz, 1H), 7.23 (t, J 6.8 Hz, 2H), 7.27 (d, J 8.4 Hz, 2H), 7.63 (d, J 8.4 Hz, 2H), 7.84 (d, J 8.4 Hz, 2H), 7.87 (s, 1H), 12.44 (br s, 1H); m/z (ES⁺) 440 (M⁺+H)⁺.

### EXAMPLE 4

### 3-cyclohexyl-1,2-diphenyl-1H-indole-6-carboxylic acid

### Step 1: methyl 2-phenyl-1H-indole-6-carboxylate

Following the procedure described in example 2 step 4, treatment of a solution (0.3 M) of methyl 3-[(trifluoroacetyl)amino]-4-{[(trifluoromethyl)sulfonyl]oxy} with ethynyl benzene (2.0 eq), tetramethyl guanidine (10.0 eq), PdCl₂(PPh₃)₂ (0.1 eq) and CuI (0.1 eq) afforded a residue that was purified by flash chromatography (1:9 AcOEt:petroleum ether eluent) to afford the title compound (39%) as a solid.

¹H NMR (400 MHz, DMSO-d₆) δ 3.88 (s, 3H), 7.04 (s, 1H), 7.40 (t, J 7.6 Hz, 1H), 7.53 (t, J 7.6 Hz, 2H), 7.65 (s, 2H), 7.92 (d, J 7.6 Hz, 2H), 8.08 (s, 1H), 11.94 (s, 1H)

### Step 2: methyl 3-cyclohex-2-en-1-yl-2-phenyl-1H-indole-6-carboxylate

Following the procedure described in example 2 step 5 treatment of a solution (0.06 M) of methyl 2-phenyl-1H-indole-6-carboxylate in dry DMF with NaH (1.1 eq) and 3-bromocyclohexene (1.3 eq) afforded a residue that was purified by flash chromatography on silica gel (1:9 AcOEt:petroleum ether) to afford the title compound (79%) as a solid.

¹H NMR (400 MHz, DMSO-d₆) δ 1.57-1.74 (m, 1H), 1.82-2.05 (m, 3H), 2.06-2.18 (m, 1H), 2.18-2.32 (m, 1H), 3.67-3.81 (m, 1H), 3.87 (s, 3H), 5.69 (d, J 10.4 Hz, 1H), 5.82-5.92 (m, 1H), 7.44-7.52 (m, 1H), 7.54-7.63 (m, 5H), 7.68 (d, J 8.4 Hz, 1H), 8.03 (s, 1H), 11.59 (s, 1H).

### Step 3: methyl 3-cyclohexyl-2-phenyl-1H-indole-6-carboxylate

A solution (0.01 M) of methyl 3-cyclohex-2-en-1-yl-2-phenyl-1H-indole-6-carboxylate in MeOH was treated with 10% Pd/C (10% wt.). The resulting suspension was stirred for 12 h under an atmosphere of hydrogen then purged with nitrogen and filtered. The filtrate was concentrated to afford the title compound (91%) as a solid.

¹H NMR (300 MHz, DMSO-d₆) δ 1.21-1.45 (m, 3H), 1.67-1.90 (m, 5H), 1.91-2.11 (m, 2H), 2.82-2.99 (m, 1H), 3.88 (s, 3H), 7.43-7.52 (m, 1H), 7.54-7.60 (m, 4H), 7.62 (dd, J 1.4, 8.4 Hz, 1H), 7.87 (d, J 8.4 Hz, 1H), 8.02 (d, J 1.4 Hz, 1H), 11.51 (s, 1H).

### Step 4: 3-cyclohexyl-1,2-diphenyl-1H-indole-6-carboxylic acid

A solution (0.05 M) of methyl 3-cyclohexyl-2-phenyl-1H-indole-6-carboxylate in toluene was treated with bromobenzene (1.2 eq) and Cs₂CO₃ (1.7 eq). Pd(P^{t}Bu₃) (0.2 eq) was added and the mixture was stirred at 100° C. for 12 h. The mixture was diluted with AcOEt then washed with brine. The dried organic phase was concentrated to give a residue that was purified by flash chromatography on silica gel (3:97 AcOEt:PE) to give a residue that was diluted to 0.03 M with a 4:1 mixture of THF:H₂O. LiOH.H₂O (12 eq) was added and the mixture was stirred at 70° C. for 3 days. Following solvent removal, the residue was treated with aqueous HCl (1 N) then filtered and purified by HPLC (stationary phase: Waters Symmetry C₁₈ 19×100 mm; mobile phase: 50% to 100% MeCN in H₂O over 10 min; retention time: 8.9 min) to give the title compound (40%) as a solid.

¹H NMR (400 MHz, DMSO-d₆) δ 1.19-1.40 (m, 3H), 1.66-1.70 (m, 1H), 1.77-1.81 (m, 4H), 1.91-2.07 (m, 2H), 2.67-2.78 (m, 1H), 7.22-7.25 (m, 4H), 7.29-7.36 (m, 4H), 7.41 (t, J 7.4 Hz, 2H), 7.70 (s, 1H), 7.71 (d, J 8.4 Hz, 1H), 7.94 (d, J 8.4 Hz, 1H), 12.55 (br s, 1H); MS (ES⁻) m/z 394 (M−H)⁻

### EXAMPLE 5

### 1-benzyl-3-cyclohexyl-2-phenyl-1H-indole-6-carboxylic acid

A solution (0.05 M) of methyl 3-cyclohexyl-2-phenyl-1H-indole-6-carboxylate in THF was treated with a suspension of 60% sodium hydride in mineral oil (1.4 eq) and the mixture was stirred for 1 h at room temperature. Following the addition of benzylbromide (1.05 eq), the mixture was stirred at 50° C. for 4 h. The solvent was removed in vacuo to give a residue that was diluted to 0.03 M with CH₂Cl₂. BBr₃ (3 eq) was added and the mixture was stirred for 2 h. Following solvent removal, the residue was treated with aqueous HCl (1 N) then filtered and purified by HPLC (stationary phase: Waters Symmetry C₁₈ 19×100 mm; mobile phase: 40% to 100% MeCN in H₂O over 11 min) to give the title compound (51%) as a solid.

¹H NMR (400 MHz, DMSO-d₆) δ 1.15-1.36 (m, 3H), 1.63-1.69 (m, 1H), 1.70-1.81 (m, 4H), 1.83-1.92 (m, 2H), 2.54-2.63 (m, 1H), 5.30 (s, 2H), 6.81 (d, J 7.2 Hz, 2H), 7.15-7.24 (m, 3H), 7.34-7.35 (m, 2H), 7.46-7.50 (m, 3H), 7.65 (d, J 8.4 Hz, 1H), 7.86 (d, J 8.4 Hz, 1H), 7.91 (s, 1H), 12.49 (br s, 1H); MS (ES⁺) m/z 410 (M+H)⁺

### EXAMPLE 6

### 3-cyclohexyl-1-(4-methylbenzyl)-2-phenyl-1H-indole-6-carboxylic acid

Following the procedure described in example 5, treatment of methyl 3-cyclohexyl-2-phenyl-1H-indole-6-carboxylate with NaH and 4-methylbenzyl bromide afforded a residue that was purified by HPLC (stationary phase: Waters Symmetry C₁₈ 19×100 mm; mobile phase: 40% to 100% MeCN in H₂O over 11 min) to give the title compound (60%) as a solid.

¹H NMR (400 MHz, DMSO-d₆) δ 1.12-1.32 (m, 3H), 1.63-1.68 (m, 1H), 1.69-1.80 (m, 4H), 1.82-1.90 (m, 2H), 2.20 (s, 3H), 2.53-2.60 (m, 1H), 5.21 (s, 2H), 6.71 (d, J 7.6 Hz, 2H), 7.02 (d, J 7.6 Hz, 2H), 7.34-7.36 (m, 2H), 7.45-7.52 (m, 3H), 7.64 (d, J 8.4 Hz, 1H), 7.8 (d, J 8.4 Hz, 1H), 7.89 (s, 1H), 12.50 (br s, 1H); MS (ES⁻) m/z 422 (M−H)⁻

### EXAMPLE 7

### 3-cyclohexyl-1-[(2-methyl-1,3-thiazol-4-yl)methyl]-2-phenyl-1H-indole-6-carboxylic acid

Following the procedure described in example 5 treatment of methyl 3-cyclohexyl-2-phenyl-1H-indole-6-carboxylate with 4-(chloromethyl)-2-methyl-1,3-thiazole afforded a residue that was purified by HPLC (stationary phase: Waters Symmetry C₁₈ 19×100 mm; mobile phase: 40% to 100% MeCN in H₂O over 11 min) to give the title compound (18%) as a solid.

¹H NMR (400 MHz, DMSO-d₆) δ 1.11-1.35 (m, 3H), 1.61-1.69 (m, 1H), 1.70-1.79 (m, 4H), 1.80-1.91 (m, 2H), 2.51-2.60 (m, 1H), 2.55 (s, 3H), 5.21 (s, 2H), 6.74 (s, 1H), 7.40-7.44 (m, 2H), 7.47-7.53 (m, 3H), 7.65 (d, J 8.4 Hz, 1H), 7.84 (d, J 8.4 Hz, 11H), 8.10 (s, 1H); MS (ES⁺) m/z 431 (M+H)⁺

### EXAMPLE 8

### 3-cyclohexyl-1-(3-methylbenzyl)-2-phenyl-1H-indole-6-carboxylic acid

Following the procedure described in example 5, treatment of methyl 3-cyclohexyl-2-phenyl-1H-indole-6-carboxylate with 3-methyl benzylbromide afforded a residue that was purified by HPLC (stationary phase: Waters Symmetry C₁₈ 19×100 mm; mobile phase: 40% to 100% MeCN in H₂O over 11 min) to give the title compound (96%) as a solid.

¹H NMR (300 MHz, DMSO-d₆) δ 1.19-1.36 (m, 3H), 1.65-1.92 (m, 7H), 2.19 (s, 3H), 2.52-2.66 (m, 1H), 5.24 (s, 2H), 6.55 (d, J 7.5 Hz, 1H), 6.70 (s, 1H), 6.99 (d, J 7.5 Hz, 1H), 7.10 (t, J 7.5 Hz, 1H), 7.35-7.38 (m, 2H), 7.49-7.55 (m, 3H), 7.66 (dd, J 8.4, 0.9 Hz, 1H), 7.87 (d, J 8.4 Hz, 1H), 7.93 (d, J 0.9 Hz, 1H); MS (ES⁺) m/z 424 (M+H)⁺

### EXAMPLE 9

### 3-cyclohexyl-2-phenyl-1-(pyridin-2-ylmethyl)-1H-indole-6-carboxylic acid trifluoroacetate

Following the procedure described in example 5, treatment of methyl 3-cyclohexyl-2-phenyl-1H-indole-6-carboxylate with 2-(chloromethyl)pyridine hydrochloride afforded a residue that was purified by HPLC (stationary phase: Waters Symmetry C₁₈ 19×100 mm; mobile phase: 10% to 90% MeCN in H₂O over 10 min) to give the title compound (59%) as a solid.

¹H NMR (400 MHz, DMSO-d₆) δ 1.12-1.30 (m, 3H), 1.61-1.68 (m, 1H), 1.70-1.78 (m, 4H), 1.81-1.91 (m, 2H), 2.55-2.62 (m, 1H), 5.31 (s, 2H), 6.67 (d, J 8.0 Hz, 1H), 7.21-7.25 (m, 1H), 7.34-7.36 (m, 2H), 7.45-7.49 (m, 3H), 7.65-7.69 (m, 2H), 7.86 (d, J 8.4 Hz, 1H), 7.90 (s, 1H), 8.45 (d, J 4.8 Hz, 1H); MS (ES⁺) m/z 411 (M+H)⁺

### EXAMPLE 10

### 3-cyclohexyl-1-[4-(methylsulfonyl)benzyl]-2-phenyl-1H-indole-6-carboxylic acid

Following the procedure described in example 5, treatment of methyl 3-cyclohexyl-2-phenyl-1H-indole-6-carboxylate with 1-(bromomethyl)-4-(methylsulfonyl)benzene afforded a residue that was purified by HPLC (stationary phase: Waters Symmetry C₁₈ 19×100 mm; mobile phase: 40% to 100% MeCN in H₂O over 11 min) to give the title compound (99%) as a solid.

¹H NMR (300 MHz, DMSO-d₆) δ 1.18-1.37 (m, 3H), 1.66-1.98 (m, 7H), 2.55-2.69 (m, 1H), 3.15 (s, 3H), 5.42 (s, 2H), 7.05 (d, J 8.4 Hz, 2H), 7.34-7.38 (m, 2H), 7.47-7.52 (m, 3H), 7.69 (dd, J 8.4, 1.2 Hz, 1H), 7.79 (d, J 8.4 Hz, 2H), 7.90 (d, J 8.4 Hz, 2H), 7.90 (d, J 8.4 Hz, 1H), 7.94 (d, J 1.2 Hz, 1H); MS (ES⁺) m/z 488 (M+H)⁺

### EXAMPLE 11

### 3-cyclohexyl-1-(3,5-dibromobenzyl)-2-phenyl-1H-indole-6-carboxylic acid

Following the procedure described in example 5, treatment of methyl 3cyclohexyl-2-phenyl-1H-indole-6-carboxylate with 3,5-dibromobenzyl bromide afforded a residue that was purified by HPLC (stationary phase: Waters Symmetry C₁₈ 19×100 mm; mobile phase: 40% to 100% MeCN in H₂O over 11 min) to give the title compound (68%) as a solid.

¹H NMR (400 Mz, DMSO-d₆) δ 1.16-1.30 (m, 3H), 1.63-1.66 (m, 1H), 1.70-1.78 (m, 4H), 1.82-1.92 (m, 2H), 2.55-2.66 (m, 1H), 5.32 (s, 2H), 6.89 (s, 1H), 6.90 (s, 1H), 7.29-7.31 (m, 2H), 7.46-7.53 (m, 3H), 7.64 (s, 1H), 7.69 (d, J 8.4 Hz, 1H), 7.88 (d, J 8.4 Hz, 2H), 8.03 (s, 1H), 12.58 (br s, 1H); MS (ES⁺) m/z 568 (M+H)⁺

### EXAMPLE 12

### 3-cyclohexyl-1-(1H-imidazol-4-ylmethyl)-2-phenyl-1H-indole-6-carboxylic acid trifluoroacetate

A solution (0.06 M) of (1-trityl-1H-imidazol-4-yl)methanol in CH₂Cl₂ was cooled at 0° C. Triethylamine (5.0 eq) and methanesulfonyl chloride (2.3 eq) were added and the mixture was stirred at 0° C. for 5 h. The mixture was diluted with CH₂Cl₂ then washed sequentially with saturated aqueous KHSO₄ and brine. The solvent was removed in vacuo to afford (1-trityl-1H-imidazol-4-yl)methyl methanesulfonate as a solid. A solution (0.03 M) of methyl 3-cyclohexyl-2-phenyl-1H-indole-6-carboxylate in DMF was treated with NaH (1.5 eq) and stirred for 1 h at room temperature. (1-Trityl-1H-imidazol-4-yl)methyl methanesulfonate (2.0 eq) was added and the mixture was stirred at 80° C. for 12 h. After dilution with AcOEt the organic phase was washed with aqueous HCl (1 N) and brine. The dried organic layer was concentrated and diluted to 0.02 M with CH₂Cl₂. BBr₃ (3 eq) was added and the mixture was stirred for 2 h. Following solvent removal, the residue was treated with aqueous HCl (1 N) then filtered and purified by HPLC (stationary phase: Waters Symmetry C₁₈ 19×100 mm; mobile phase: 10% to 90% MeCN in H₂O over 10 min; retention time: 9.0 min) to give the title compound (54%) as a solid.

¹H NMR (400 MHz, DMSO-d₆) δ 1.17-1.31 (m, 3H), 1.65-1.74 (m, 5H), 1.81-1.90 (m, 2H), 2.53-2.60 (m, 1H), 5.32 (s, 2H), 6.93 (s, 1H), 7.36-7.42 (m, 2H), 7.49-7.56 (m, 3H), 7.71 (d, J 8.4 Hz, 1H), 7.89 (d, J 8.4 Hz, 1H), 8.07 (s, 1H), 8.85 (s, 1H); MS (ES⁺) m/z 400 (M+H)⁺

### EXAMPLE 13

### 3-cyclohexyl-2-phenyl-1-(pyridin-3-ylmethyl)-1H-indole-6-carboxylic acid hydrochloride

Following the procedure described in example 5, treatment of methyl 3-cyclohexyl-2-phenyl-1H-indole-6-carboxylate with (3-bromomethyl)pyridine hydrobromide afforded a residue that was purified by HPLC (stationary phase: Waters Symmetry C1₈ 19×100 mm; mobile phase: 40% to 100% MeCN in H₂O over 11 min) to give the title compound (36%) as a solid.

¹H NMR (400 MHz, DMSO-d₆) δ 1.12-1.31 (m, 3H), 1.63-1.68 (m, 1H), 1.70-1.78 (m, 4H), 1.81-1.92 (m, 2H), 2.53-2.62 (m, 1H), 5.44 (s, 2H), 7.32-7.35 (m, 2H), 7.43 (d, J 7.6 Hz, 1H), 7.47-7.51 (m, 3H), 7.52-7.56 (m, 1H), 7.69 (d, J 8.4 Hz, 1H), 7.89 (d, J 8.4 Hz, 1H), 8.04 (s, 1H), 8.20 (s, 1H), 8.55 (d, J 5.5 Hz, 1H); MS (ES⁺) m/z 411 (M+H)⁺

### ADDITIONAL EXAMPLES

| Name | Structure | Molecular Ion [M + H]⁺ |
|---|---|---|
| 3-cyclohexyl-2-(2-fluorophenyl)-1-(2-phenyl- ethyl)-1H-indole-6-carboxylic acid | 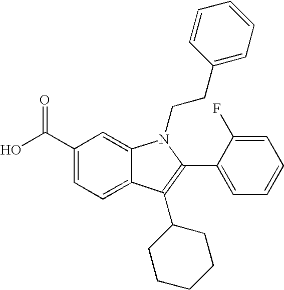 | 442 |
| 1-(3-cyanobenzyl)-3-cyclohexyl-2-phe- nyl-1H-indole-6-carboxylic acid | 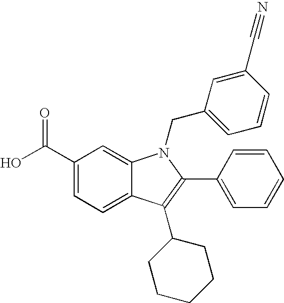 | 435 |
| 3-cyclohexyl-2-phenyl-1-(pyridin-2-yl- methyl)-1H-indole-6-carboxylic acid hydrochloride | 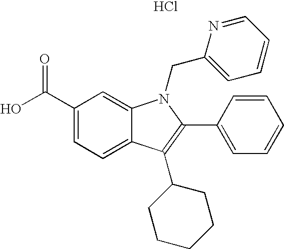 | 411 |
| 1-(3-carboxybenzyl)-3-cyclohexyl-2-phe- nyl-1H-indole-6-carboxylic acid | 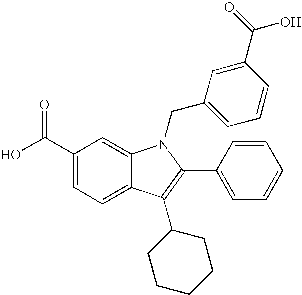 | 454 |
| 3-cyclohexyl-2-(4-hydroxyphenyl)-1-[(4-meth- ylphenyl)sulfonyl]-1H-in- dole-6-carboxylic acid | 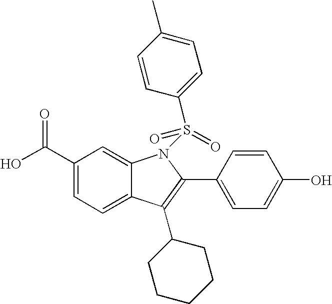 | 490 |
| 1-benzoyl-3-cyclohexyl-2-phenyl-1H-in- dole-6-carboxylic acid | 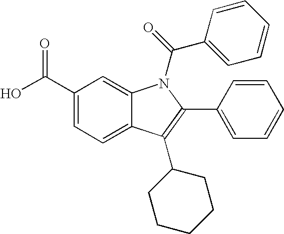 | 424 |
| 3-cyclohexyl-2-phenyl-1-(phenyl- sulfonyl)-1H-indole-6-car- boxylic acid | 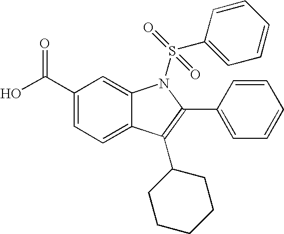 | 460 |
| 1-benzyl-3-cyclohexyl-2-(3-{[iso- propyl(methyl)amino]-meth- yl}phenyl)-1H-indole-6-car- boxylic acid | 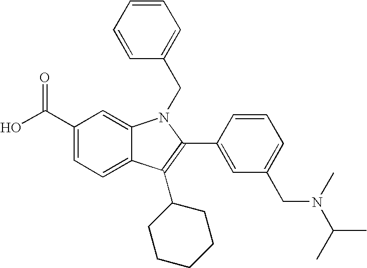 | 495 |
| 3-cyclohexyl-1-({5-[(di- methylamino)methyl]-1,2,4-oxa- diazol-3-yl}methyl)-2-phenyl-1-1H-in- dole-6-carboxylic acid | 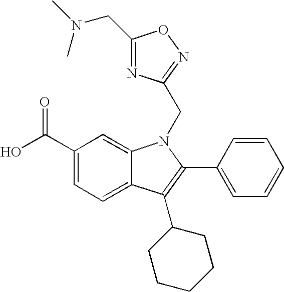 | 459 |

1. A compound of formula (I), or a pharmaceutically acceptable salt thereof: 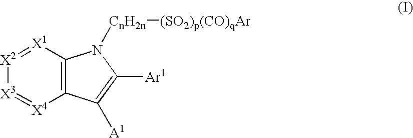
wherein:
Ar is a moiety containing at least one aromatic ring and possesses 5-, 6-, 9- or 10-ring atoms 0 to 3 of which may be N, O or S heteroatoms of which at most 1 will be O or S; which moiety may be optionally substituted by groups Q¹, Q² or Q³ wherein Q¹ is a hydroxy group, fluorine, chlorine, bromine or iodine atom or a C₁₋₆alkyl, C₁₋₆alkyl substituted by not more than 5 fluorine atoms, C₁₋₆alkoxyl, C₁₋₆alkoxyl substituted by not more than 5 fluorine atoms, C₂₋₆alkenyl, C₂₋₆alkynyl, (CH₂)₀₋₃N(C₁₋₄alkyl)₂, nitro, cyano, nitrile, carboxyl, esterified carboxy wherein the esterifying moiety has up to 4 carbon atoms optionally substituted by not more than 5 fluorine atoms; or —SO₂(C₁₋₆alkyl), Q₂ is a fluorine, chlorine, bromine or iodine atom or a methyl, trifluoromethyl, methoxy, trifluoromethoxy or difluoromethoxy group, Q₃ is a fluorine, chlorine, bromine or iodine atom or a methyl, methoxy, trifluoromethoxy or difluoromethoxy group;
Ar¹ is a moiety containing at least one aromatic ring and possesses 5-, 6-, 9- or 10-ring atoms 0 to 3 of which may be N, O or S heteroatoms of which at most 1 will be O or S; which moiety may be optionally substituted by groups Q⁴, Q⁵ or Q⁶ wherein Q⁴ is a hydroxy group, fluorine, chlorine, bromine or iodine atom or a C₁₋₆alkyl, C₁₋₆alkyl substituted by not more than 5 fluorine atoms, C₁₋₆alkoxyl, C₁₋₆alkoxyl substituted by not more than 5 fluorine atoms, C₂₋₆alkenyl or alkynyl, nitro, cyano, nitrile, carboxyl, esterified carboxy wherein the esterifying moiety has up to 4 carbon atoms optionally substituted by not more than 5 fluorine atoms, Q⁵ is a fluorine, chlorine, bromine or iodine atom or a methyl, trifluoromethyl, methoxy, trifluoromethoxy or difluoromethoxy group, Q⁶ is a fluorine, chlorine, bromine or iodine atom or a methyl, methoxy, trifluoromethoxy or difluoromethoxy group;
X is Nor CR^{a}; X²is N or CR¹; X³ is N or CR²; X⁴ is N or CR^{b}; with the proviso that at least one of X² and X³ is not N; wherein R^{a} and R^{b} are independently selected from hydrogen, fluorine or chlorine or C₁₋₄alkyl, C₂₋₄alkenyl, C₁₋₄alkoxy, C₁₋₄alkyl or alkoxy optionally substituted by up to 6 fluorine atoms and/or a hydroxyl group;
n is 0, 1, 2, 3, 4, 5 or 6;
p+q is 0 or 1;
A¹ is C₁₋₆alkyl, C₂₋₆alkenyl, or C₁₋₆alkyl or C₂₋₆alkenyl substituted by C₁₋₄alkoxy or up to 5 fluorine atoms or a non-aromatic ring of 3 to 8 ring atoms which may contain a double bond and which may contain a O, S, SO, SO₂ or NH moiety and which may be optionally substituted by one or two alkyl groups of up to 2 carbon atoms or by 1 to 8 fluorine atoms;
one of R¹ and R² is a Het or is hydrogen, fluorine, chlorine or bromine atom or a C₁₋₄alkyl, C₂₋₄alkenyl, C₁₋₄alkoxy, C₁₋₄alkyl or alkoxy substituted by up to 5 fluorine atoms, nitrile, carboxy, C₁₋₄alkoxycarbonyl, C₁₋₄alkyl or C₂₋₄alkenyl substituted by a carboxy or C₁₋₄alkoxycarbonyl group, or a NR³R⁴, SO₂NR³R⁴ or CONR³R⁴ group where R³ is hydrogen, C₁₋₄alkyl, SO₂R⁵ or COR⁵ and R⁴ is hydrogen, hydroxyl or C₁₋₄alkyl or R³ and R⁴ are alkylene linked to form a 5- or 6-membered ring, and R⁵ is C₁₋₄alkyl optionally substituted by up to 5 fluorine atoms;
Het is a 5 or 6-membered aromatic ring of which 1, 2, 3 or 4 ring atoms may be selected from N, O, S with at most 1 being O or S which ring may be substituted by 1 or 2 groups selected C₁₋₄alkyl or hydroxy or tautomers thereof, or is 2-hydroxy-cyclobutene-3,4-dione;
the other of R¹ and R² is a hydrogen, fluorine or chlorine atom or C₁₋₄alkyl, C₂₋₄alkenyl, C₁₋₄alkoxy, C₁₋₄alkyl or alkoxy substituted by up to 6 fluorine atoms and optionally a hydroxyl.

2. A compound as claimed in claim 1 wherein R^{a} is hydrogen.

3. A compound as claimed in claim 2 wherein R^{b} is hydrogen.

4. A compound as claimed in claim 3 wherein Ar is optionally substituted phenyl, pyridyl, imidazolyl, thiazolyl or oxadiazolyl, where the optional substituent is selected from fluorine, chlorine, bromine, C₁₋₆alkyl, hydroxyl, C₁₋₆alkoxy, CF₃, cyano, carboxyl, methylsulfonyl and (CH₂)₀₋₃N(C₁₋₄alkyl)₂.

5. A compound as claimed in claim 4 wherein n is 0, 1 or 2.

6. A compound as claimed in claim 5 wherein Ar¹ is phenyl, naphthyl, indolyl, tetrahydronaphthyl, pyridyl, imidazolyl, furyl, thienyl, pyrolidyl, oxazolyl, thiazolyl, pyrazolyl, pyridazolyl, triazolyl, oxadiazolyl, thiodiazolyl or quinonyl, optionally substituted by Q⁴, Q⁵ or Q⁶ as defined in claim 1.

7. A compound as claimed in claim 6 wherein Ar is cyclohexyl.

8. A compound as claimed in claim 1 of formula (II): 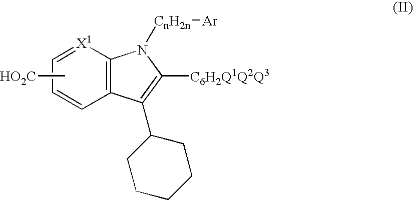
or a pharmaceutically acceptable salt thereof.

9. A compound according to claim 8 of formula (III): 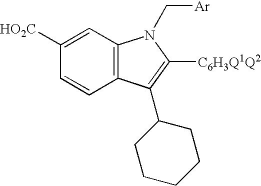
or a pharmaceutically acceptable salt thereof.

10. A compound as claimed in claim 1 selected from:
1-benzyl-3-cyclohexyl-2-phenyl-1H-indole-5-carboxylic acid,
1-benzyl-3-cyclohexyl-2-pyridin-2-yl-1H-indole-6-carboxylic acid,
1-benzyl-3-cyclohexyl-2-(4-methoxyphenyl)-1H-indole-6-carboxylic acid,
3-cyclohexyl-1,2-diphenyl-1H-indole-6-carboxylic acid,
1-benzyl-3-cyclohexyl-2-phenyl-1H-indole-6-carboxylic acid,
3-cyclohexyl-1-(4-methylbenzyl)-2-phenyl-1H-indole-6-carboxylic acid,
3-cyclohexyl-1-[(2-methyl-1,3-thiazol-4-yl)methyl]-2-phenyl-1H-indole-6-carboxylic acid,
3-cyclohexyl-1-(3-methylbenzyl)-2-phenyl-1H-indole-6-carboxylic acid,
3-cyclohexyl-2-phenyl-1-(pyridin-2-ylmethyl)-1H-indole-6-carboxylic acid trifluoroacetate,
3-cyclohexyl-1-[4-(methylsulfonyl)benzyl]-2-phenyl-1H-indole-6-carboxylic acid,
3-cyclohexyl-1-(3,5-dibromobenzyl)-2-phenyl-1H-indole-6-carboxylic acid,
3-cyclohexyl-1-(1H-imidazol-4-ylmethyl)-2-phenyl-1H-indole-6-carboxylic acid trifluoroacetate,
3-cyclohexyl-2-phenyl-1-(pyridin-3-ylmethyl)-1H-indole-6-carboxylic acid hydrochloride,
3-cyclohexyl-2-(2-fluorophenyl)-1-(2-phenylethyl)-1H-indole-6-carboxylic acid,
1-(3-cyanobenzyl)-3-cyclohexyl-2-phenyl-1H-indole-6-carboxylic acid,
3-cyclohexyl-2-phenyl-1-(pyridin-2-ylmethyl)-1H-indole-6-carboxylic acid hydrochloride,
1-(3-carboxybenzyl)-3-cyclohexyl-2-phenyl-1H-indole-6-carboxylic acid,
3-cyclohexyl-2-(4-hydroxyphenyl)-1-[(4-methylphenyl)sulfonyl]-1H-indole-6-carboxylic acid,
1-benzoyl-3-cyclohexyl-2-phenyl-1H-indole-6-carboxylic acid,
3-cyclohexyl-2-phenyl-1-(phenylsulfonyl)-1H-indole-6-carboxylic acid,
1-benzyl-3-cyclohexyl-2-(3-{[isopropyl(methyl)amino]-methyl}phenyl)-1H-indole-6-carboxylic acid,
3-cyclohexyl-1-({5-[(dimethylamino)methyl]-1,2,4-oxadiazol-3-yl}methyl)-2-phenyl-1-1H-indole-6-carboxylic acid,
or a pharmaceutically acceptable salt thereof.

11. (canceled)

12. (canceled)

13. A pharmaceutical composition comprising a compound as claimed in claim 1, or a pharmaceutically acceptable salt thereof, in association with a pharmaceutically acceptable carrier.

14. The pharmaceutical composition as claimed in claim 13 which further comprises one or more other agents for the treatment of viral infections.

15. A method of inhibiting hepatitis C virus polymerase and/or of treating or preventing an illness due to hepatitis C virus, which comprises administering to a human or animal subject suffering from the condition a therapeutically or prophylactically effective amount of a compound as claimed in claim 1, or a pharmaceutically acceptable salt thereof.

16. A method of preparation of a pharmaceutical composition, which comprises admixing at least one compound as claimed in claim 1, or a pharmaceutically acceptable salt thereof, with one or more pharmaceutically acceptable adjuvants, diluents or carriers and/or with one or more other therapeutically or prophylactically active agents.

17. A process to prepare a compound as claimed in claim 1 which comprises the reaction of compounds of the formulae (IV) and (V): 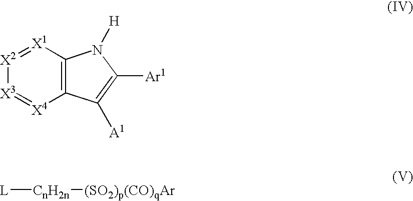
wherein X¹, X², X³, X⁴, A¹, Ar¹, Ar, n, p and q are as defined in claim 1 and L is a leaving group.

18. A process to prepare a compound as claimed in claim 1 which comprises reacting the compound of the formula (VI): 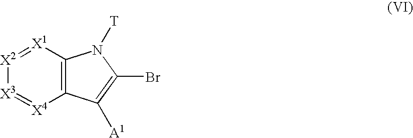
wherein T is a CₙH₂ₙ(SO₂)ₚ(CO)_{q}Ar group with Ar¹B(OH)₂ in the presence of a Pd[0] catalyst wherein X¹, X², X³, X⁴, A¹, Ar¹, Ar, n, p and q are as defined in claim 1.

19. A method for inhibiting hepatitis C virus polymerase or treating or preventing an illness due to hepatitis C virus, which comprises administering to a human or animal subject suffering from the condition a therapeutically or prophylactically effective amount of the pharmaceutical composition of claim 13.
